(19) United States
(12) Patent Application Publication
(10) Pub. No.: US 2025/0255746 A1
(43) Pub. Date: Aug. 14, 2025

(54) Device for Stimulating an Erection

(71) Applicant: Dongguan Sena Toys Co., Ltd., Dongguan (CN)

(72) Inventor: Rui Li, Dongguan (CN)

(21) Appl. No.: 19/193,843

(22) Filed: Apr. 29, 2025

(51) Int. Cl. A61F 5/41

(52) U.S. Cl. CPC A61F 5/41; A61F 2005/412

(57) ABSTRACT

A device for stimulating an erection includes a soft sheath, a structure simulating the human oral cavity and an aperture. The aperture is arranged in the axial direction of the soft sheath. The structure simulating the human oral cavity is arranged inside the aperture. A male's penis can be inserted into the structure simulating the human oral cavity along the aperture, and the penis will feel a sense of being wrapped by the oral cavity, so as to generate strong sexual stimulation.

### TECHNICAL FIELD

The invention belongs to the technical field of sexual devices, and particularly relates to a device for stimulating an erection.

### TECHNICAL BACKGROUND

There are many male sexual stimulation devices known in the prior art. The existing male sexual stimulation devices are mainly a combination of a silicone sheath and a pump. The pump is used to generate negative pressure to promote blood circulation, so as to treat male erectile dysfunction.

However, the structure and mode of the existing sexual stimulation devices are relatively simple, men will gradually lose interest in the sexual stimulation devices after long-term use. Therefore, it is necessary to propose a sexual stimulation device with different experiences.

### INVENTION CONTENT

In order to overcome the shortcomings of the prior art, the present invention provides a device for stimulating an erection, which includes a soft sheath. A structure simulating a human oral cavity and an aperture are arranged inside the soft sheath. A male's penis can be inserted into the structure simulating a human oral cavity along the aperture, and the penis will feel a sense of being wrapped by the oral cavity, so as to generate strong sexual stimulation.

The technical scheme adopted by the present invention for solving the technical problems is as follows:

A device for stimulating an erection includes a soft sheath. An aperture is arranged in the axial direction of the soft sheath, and a structure simulating a human oral cavity is arranged inside the aperture.

Further, the soft sheath is provided with a first soft member, a second soft member and a third soft member. The first soft member is arranged at one end of the soft sheath. The second soft member is arranged on two opposite inner walls of the aperture. The third soft member is arranged on a side of the second soft member away from the first soft member.

Further, a first gap is arranged between the first soft member and the second soft member. A second gap is arranged between the second soft member and the third soft member.

Further, a bulge structure is arranged on a side of the third soft member away from the second soft member. A plurality of threads are arranged on a surface of the bulge structure.

Further, there are two bulge structures. One bulge structure is arranged on an upper wall of the aperture, and other bulge structure is arranged on a lower wall of the aperture. The two bulge structures are arranged alternately.

Further, a plurality of ring structures at even intervals are also arranged on the inner wall of the aperture. The plurality of ring structures is arranged on a side of the bulge structure away from the third soft member.

Further, the device also includes a negative pressure cylinder and an air pumping structure for vacuuming the negative pressure cylinder. One end of the negative pressure cylinder is covered by the soft sheath; other end of the negative pressure cylinder is provided with the air pumping structure.

Further, an annular groove is arranged at one end of the soft sheath away from the first soft member, and the annular groove is adapted to the cylinder wall of the negative pressure cylinder.

Further, the air pumping structure adopts an electric pump, and a plurality of charging contacts are arranged on the electric pump.

Further, the air pumping structure adopts a manual pump. The manual pump is connected to the negative pressure cylinder through a conduit and a valve.

The beneficial effects of the present invention:

The present invention provides a device for stimulating an erection. The device for stimulating an erection includes a soft sheath. A structure simulating a human oral cavity and an aperture are arranged inside the soft sheath. A male's penis can be inserted into the structure simulating a human oral cavity along the aperture, and the penis will feel a sense of being wrapped by the oral cavity, so as to generate strong sexual stimulation.

### DESCRIPTION OF FIGURES

FIG. 1 is a schematic view of a device for stimulating an erection in Embodiment I of the present invention;

FIG. 2 is a schematic view of the device for stimulating an erection in Embodiment I of the present invention from another angle;

FIG. 3 is an exploded view of the device for stimulating an erection in Embodiment I of the present invention;

FIG. 4 is a cross-sectional view of the soft sheath of the present invention;

FIG. 5 is a schematic view of a device for stimulating an erection in Embodiment II of the present invention;

FIG. 6 is a schematic view of a device for stimulating an erection in Embodiment III of the present invention;

FIG. 7 is a schematic view of a device for stimulating an erection in Embodiment IV of the present invention;

### DESCRIPTION OF MARKS IN FIGURES

1—soft sheath; 11—aperture; 12—first soft member; 13—second soft member; 14—third soft member; 15—bulge structure; 151—thread; 16—ring structure; 17—annular groove; 2—negative pressure cylinder; 21—tick mark; 3—electric pump; 31—charging contact; 32—button; 4—manual pump; 41—conduit; 42—valve; 43—grip.

### DETAILED DESCRIPTION OF THE EMBODIMENTS

The present embodiment only shows an explanation of the present invention, and it is not a limitation to the present invention. The skilled in the art can make modifications to this embodiment as needed without making any creative contributions after reading this specification, which are always protected by the patent law as long as they are within the scope of the claims of the present invention.

It should be noted that when an element is called as being “fixed to” or “arranged on” another element, it can be directly on the other element or indirectly on the other element. When an element is called as being “connected to” another element, it can be directly connected to the other element or indirectly connected to the other element.

It should be noticed that the terms “length”, “width”, “above”, “below”, “front”, “rear”, “left”, “right”, “vertical”, “horizontal”, “top”, “bottom”, “inside” and “outside” which indicates the orientations or positional relationships are based on the orientations or positional relationships shown in the figures. They are only for facilitating describing the present invention and simplifying the description, rather than indicating or implying that the device or component must have a specific orientation, construct and operate in a specific orientation. Therefore, it cannot be understood as a limitation of the present invention.

In addition, the terms “first” and “second” are used for descriptive purposes only and cannot be understood as indicating or implying relative importance or implicitly indicating the quantity of indicated technical features. Therefore, a feature defined as “first” and “second” may explicitly or implicitly include one or more of these features. In the description of the present invention, “a plurality of” means two or more, unless otherwise specifically defined.

### Embodiment I

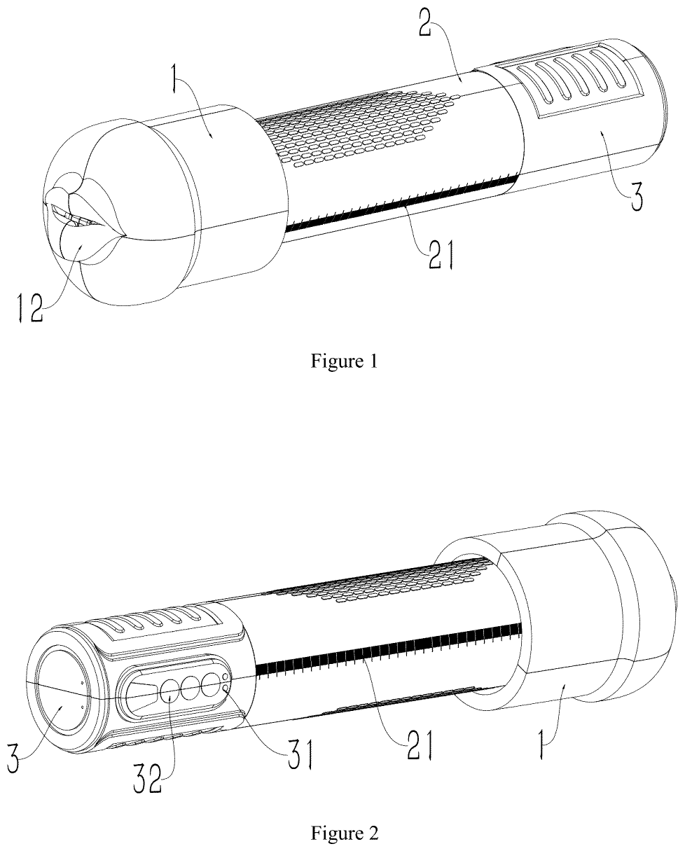

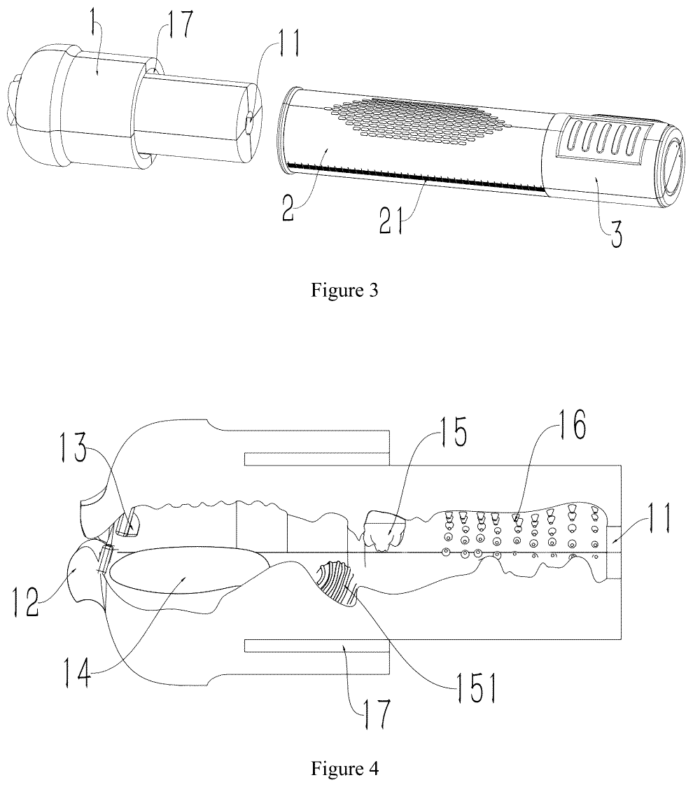

Referring to FIGS. 1-4, this embodiment provides a device for stimulating an erection, including a soft sheath 1. An aperture 11 is arranged in the axial direction of the soft sheath 1, and a structure simulating a human oral cavity is arranged inside the aperture 11.

Specifically, the soft sheath 1 is provided with a first soft member 12, a second soft member 13 and a third soft member 14. The first soft member 12 is arranged at one end of the soft sheath 1. The second soft member 13 is arranged on the two opposite inner walls of the aperture 11. The third soft member 14 is arranged on a side of the second soft member 13 away from the first soft member 12.

Preferably, a first gap is arranged between the first soft member 12 and the second soft member 13, and a second gap is arranged between the second soft member 13 and the third soft member 14.

In this embodiment, the first soft member 12 and the third soft member 14 are made of the same material, and the hardness of the second soft member 13 is greater than that of the first soft member 12.

Specifically, the first soft member 12 is configured to simulate the structure of human lips. The second soft member 13 is configured to simulate the tooth-like structure of human teeth and is symmetrically distributed up and down. The third soft member 14 is configured to simulate the structure of human tongue and is arranged on an inner wall below the aperture 11 and extends in the horizontal direction.

Understandably, the first soft member 12, the second soft member 13 and the third soft member 14 are arranged in sequence along the aperture 11. The penis can be inserted into the soft sheath 1 along the end of the soft sheath 1 provided with the first soft member 12. The first soft member 12, the second soft member 13 and the third soft member 14 cooperate together to simulate the structure of the human oral cavity, providing the penis with a sense of being wrapped by the oral cavity, so as to generate strong sexual stimulation.

Preferably, a bulge structure 15 is arranged on the side of the third soft member 14 away from the second soft member 13, and a plurality of threads 151 are arranged on the surface of the bulge structure 15.

Further, there are two bulge structures 15. One bulge structure 15 is arranged on the upper wall of the aperture 11, the other bulge structure 15 is arranged on the lower wall of the aperture 11. And the two bulge structures 15 are arranged alternately.

Further, a plurality of ring structures 16 at even intervals are further arranged on the inner wall of the aperture 11. The plurality of ring structures 16 are arranged on a side of the bulge structure 15 away from the third soft member 14. When the penis is inserted into the soft sheath 1, the two bulge structures 15 and the plurality of ring structures 16 will abut against the surface of the penis, generating a squeezing force to provide stimulation to different parts of the penis.

Preferably, the device for stimulating an erection provided in this embodiment also includes a negative pressure cylinder 2 and an air pumping structure for vacuuming the negative pressure cylinder 2. One end of the negative pressure cylinder 2 is covered by the soft sheath 1; other end of the negative pressure cylinder 2 is provided with the air pumping structure.

Specifically, an annular groove 17 is arranged at one end of the soft sheath 1 away from the first soft member 12. The annular groove 17 is adapted to the cylinder wall of the negative pressure cylinder 2, and the cylinder wall can be embedded in the annular groove 17 to fix the soft sheath 1 on the negative pressure cylinder 2.

Preferably, tick marks 21 are arranged on the outer circumference of the negative pressure cylinder 2. The user can observe the length of the penis when it is erect through the tick marks 21.

Understandably, when using the device provided in this embodiment, the user holds the negative pressure cylinder 2 with a hand and inserts the penis into the soft sheath 1 and the negative pressure cylinder 2 in sequence, and finally activates the air pumping structure to form a vacuum environment in the negative pressure cylinder 2. In this process, the arteries and veins can be expanded, and blood is slowly forced to pass through the male sexual organs, so that the length and circumference of the organs become larger, so as to stimulate the penis to erect, and restore and enhance the muscle growth and strength of the male penis. The use process of the device provided in this embodiment takes about 25 to 30 minutes. After the use time is reached, the air pumping structure is closed, and the enlarged penis is taken out from the negative pressure cylinder 2, the stimulation erection process is completed.

In actual application, the user can put a contraction ring on the penis to keep the penis in an erect state after the stimulation. Men can solve the problem of erectile dysfunction by regularly using the device provided by this embodiment to stimulate the penis and enjoy the pleasure of sexual intercourse again.

Preferably, the air pumping structure includes an electric pump 3. A plurality of buttons 32 are arranged on the electric pump 3. The operation frequency and intensity of the electric pump 3 can be adjusted by the buttons 32 to provide different modes for the user.

Further, a plurality of charging contacts 31 are arranged on the electric pump 3. The electric pump 3 can be charged through the plurality of charging contacts 31.

### Embodiment II

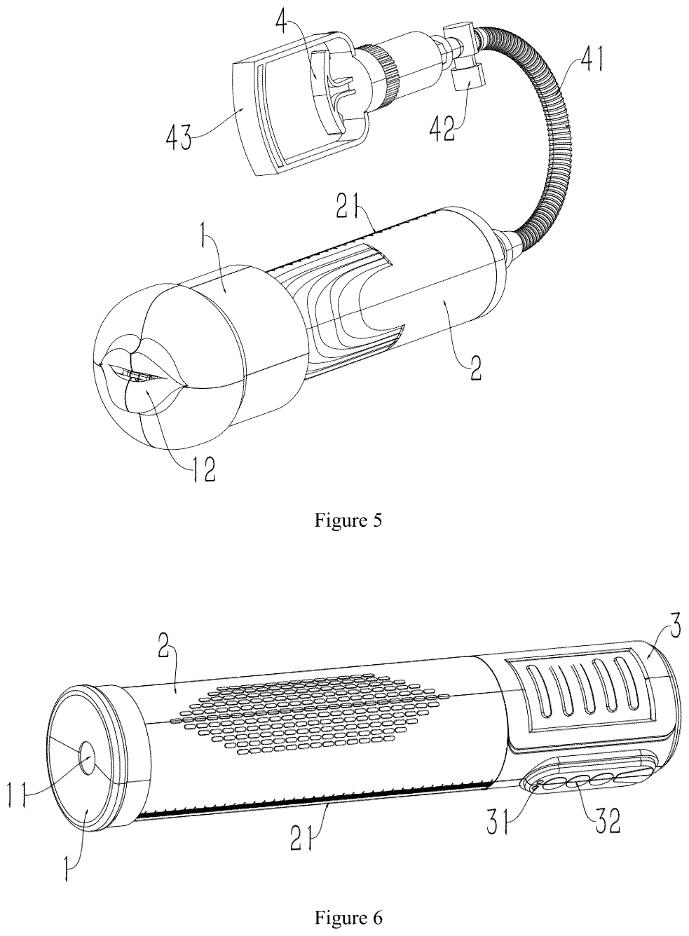

Referring to FIG. 5, the difference between this embodiment and the Embodiment I is that:

The air pumping structure of this embodiment is a manual pump 4. The manual pump 4 is connected to the negative pressure cylinder 2 through a conduit 41 and a valve 42. The valve 42 can be used as a pressure relief valve. When the negative pressure environment in the negative pressure cylinder 2 reaches a threshold, it may cause discomfort to the penis. The valve 42 can be opened to adjust the pressure relief to provide the user with a more comfortable experience.

Further, the air pumping structure also includes a grip 43. The grip 43 cooperates with the air pumping end of the manual pump 4 to facilitate the user to hold the manual pump 4 and apply force on the manual pump 4.

The remaining features and advantages of this embodiment are the same as those of the Embodiment I.

### Embodiment III

Referring to FIG. 6, the difference between this embodiment and the Embodiment I is that:

the structures of the soft sheath 1 are different.

The soft sheath 1 in this embodiment is configured as a cylindrical structure. An aperture 11 is arranged in the middle of the soft sheath 1. The soft sheath 1 is directly sleeved on the end of the negative pressure cylinder 2.

The remaining features and advantages of this embodiment are the same as those of the Embodiment I.

### Embodiment IV

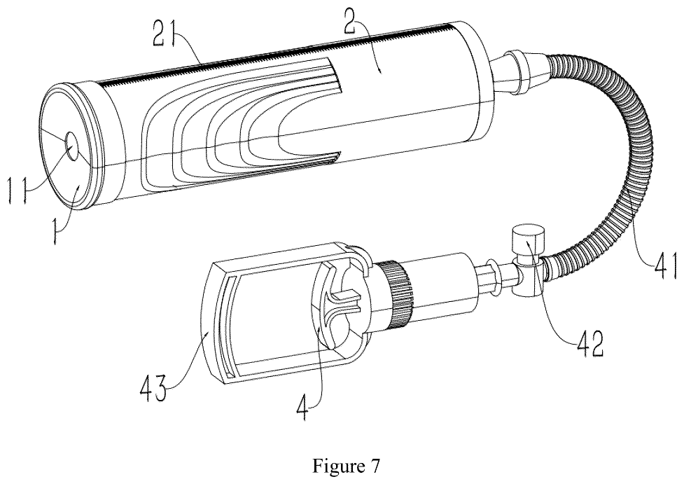

Referring to FIG. 7, the difference between this embodiment and the Embodiment I is that:

The air pumping structure of this embodiment is a manual pump 4. The soft sheath 1 is configured as a cylindrical structure with an aperture 11 in the middle.

The remaining features and advantages of this embodiment are the same as those of the Embodiment I.

It could be understood that under the guidance of the above embodiments, those skilled in the filed can combine various implementation methods in the above embodiments to obtain technical solutions of multiple implementation methods.

The above description is only a preferred embodiment of the present invention and is not to limit the present invention. Any modifications, equivalent substitutions and improvements made within the spirit and principles of the present invention should be included in the protection scope of the present invention.

1. A device for stimulating an erection comprises a soft sheath, wherein an aperture is arranged in the axial direction of the soft sheath; a structure simulating a human oral cavity is arranged inside the aperture.

2. A device for stimulating an erection according to claim 1, wherein the soft sheath is provided with a first soft member, a second soft member and a third soft member; the first soft member is arranged at one end of the soft sheath; the second soft member is arranged on two opposite inner walls of the aperture; the third soft member is arranged on a side of the second soft member away from the first soft member.

3. A device for stimulating an erection according to claim 2, wherein a first gap is arranged between the first soft member and the second soft member; a second gap is arranged between the second soft member and the third soft member.

4. A device for stimulating an erection according to claim 2, wherein a bulge structure is arranged on a side of the third soft member away from the second soft member; a plurality of threads is arranged on the surface of the bulge structure.

5. A device for stimulating an erection according to claim 4, wherein there are two bulge structures; one bulge structure is arranged on an upper wall of the aperture, and the other bulge structure is arranged on a lower wall of the aperture; the two bulge structures are arranged alternately.

6. A device for stimulating an erection according to claim 4, wherein a plurality of ring structures at even intervals are also arranged on the inner wall of the aperture;
the plurality of ring structures is arranged on a side of the bulge structure away from the third soft member.

7. A device for stimulating an erection according to claim 2, also comprises a negative pressure cylinder and an air pumping structure for vacuuming the negative pressure cylinder, wherein one end of the negative pressure cylinder is covered by the soft sheath; other end of the negative pressure cylinder is provided with the air pumping structure.

8. A device for stimulating an erection according to claim 7, wherein an annular groove is arranged at one end of the soft sheath away from the first soft member; the annular groove is adapted to the cylinder wall of the negative pressure cylinder.

9. A device for stimulating an erection according to claim 7, wherein the air pumping structure adopts an electric pump, and a plurality of charging contacts are arranged on the electric pump.

10. A device for stimulating an erection according to claim 7, wherein the air pumping structure adopts a manual pump, and the manual pump is connected to the negative pressure cylinder through a conduit and a valve.
